(19) United States
(12) Patent Application Publication
(10) Pub. No.: US 2024/0069041 A1
(43) Pub. Date: Feb. 29, 2024

(54) METHODS FOR TREATING AND MONITORING FRONTOTEMPORAL DEMENTIA

(71) Applicant: Denali Therapeutics Inc., South San Francisco, CA (US)

(72) Inventors: Giuseppe Astarita, South San Francisco, CA (US); Sarah L. Devos, South San Francisco, CA (US); Gilbert Di Paolo, South San Francisco, CA (US); Meng Fang, South San Francisco, CA (US); Fen Huang, South San Francisco, CA (US); Todd P. Logan, South San Francisco, CA (US); Matthew J. Simon, South San Francisco, CA (US)

(73) Assignee: Denali Therapeutics Inc., South San Francisco, CA (US)

(21) Appl. No.: 18/248,818

(22) PCT Filed: Oct. 13, 2021
(86) PCT No.: PCT/US2021/071835

§ 371 Date: Apr. 12, 2023

Related U.S. Application Data
(60) Provisional application No. 63/182,567, filed on Apr. 30, 2021.
(60) Provisional application No. 63/091,815, filed on Oct. 14, 2020.

(51) Int. Cl. G01N 33/68; C07K 16/28

(52) U.S. Cl. CPC G01N 33/6896; G01N 33/6848; C07K 16/286; G01N 2800/2814; A61K 2039/505

(57) ABSTRACT

The present disclosure provides methods and materials for screening a compound or monitoring a subjects response to a compound or dosing regimen for treating frontotemporal dementia (FTD). Methods and materials for identifying and treating a subject having FTD are also provided.

### CROSS-REFERENCE TO RELATED APPLICATIONS

This application claims priority to U.S. Provisional Application No. 63/091,815, filed Oct. 14, 2020, and U.S. Provisional Application No. 63/182,567, filed Apr. 30, 2021, the disclosures of which are hereby incorporated by reference in their entirety for all purposes.

### BACKGROUND

Frontotemporal dementia (FTD) is a progressive neurodegenerative disorder which accounts for 5-10% of all patients with dementia and 10-20% of patients with an onset of dementia before 65 years (Rademakers et al., Nat Rev Neurol. 8(8):423-34, 2012). While several genes have been linked to FTD, one of the most frequently mutated genes in FTD is GRN, which maps to human chromosome 17q21 and encodes the cysteine-rich protein progranulin (PGRN) (also known as proepithelin and acrogranin). Highly-penetrant mutations in GRN were first reported in 2006 as a cause of autosomal dominant forms of familial FTD (Baker et al., Nature. 442(7105):916-9, 2006; Cruts et al., Nature. 2006 Aug. 24; 442(7105):920-4; Gass et al., Hum. Mol. Genet. 15(20):2988-3001, 2006). Recent estimates suggest that GRN mutations account for 5-20% of FTD patients with positive family history and 1-5% of sporadic cases (Rademakers et al., supra). There is a need for new methods for evaluating compounds and related therapies for treating FTD, including monitoring patient responses to such treatment, as well as for identifying subjects who can benefit from treatment of these disorders.

### SUMMARY

In one aspect, the present disclosure provides a method for identifying a subject having, or at risk of having, frontotemporal dementia (FTD), the method comprising:
(a) measuring the abundance of glucosylsphingosine (GlcSph) in a test sample from a subject;
(b) comparing the difference in abundance between the GlcSph measured in (a) and one or more reference values; and
(c) determining from the comparison whether the subject has, or is at risk of having, FTD.

In some embodiments of this aspect, the method further comprises administering to the subject a compound for improving the GlcSph level for treating FTD.

In another aspect, the present disclosure provides a method for evaluating a compound or monitoring a subject's response to a compound, pharmaceutical composition, or dosing regimen thereof for treating FTD, the method comprising:
(a) measuring the abundance of GlcSph in a test sample from a subject having FTD, wherein the test sample or subject has been treated with the compound or pharmaceutical composition thereof;
(b) comparing the difference in abundance between the GlcSph measured in (a) and one or more reference values; and
(c) determining from the comparison whether the compound, pharmaceutical composition, or dosing regimen thereof improves the GlcSph level for treating FTD.

In some embodiments of this aspect, the method further comprises treating another test sample or subject with another compound and selecting a candidate compound that improves the GlcSph level.

In some embodiments of this aspect, the method further comprises:
(d) maintaining or adjusting the amount or frequency of administration of the compound to the test sample or to the subject; and
(e) administering the compound to the test sample or to the subject.

In some embodiments of the methods described herein, the compound is a sortilin inhibitor. In certain embodiments, the sortilin inhibitor is an anti-sortilin antibody.

In some embodiments, a subject having, or at risk of having, FTD has an increased GlcSph level compared to the reference value. In certain embodiments, the abundance of the GlcSph in the test sample of a subject having, or at risk of having, FTD is at least about 1.2-fold to about 5-fold higher compared to the reference value. In some embodiments, the improved GlcSph level is an improvement over the GlcSph level prior to treatment, and the improved GlcSph level is closer to the reference value than the GlcSph level prior to treatment. In particular embodiments, the improved GlcSph level has a difference compared to the reference value of less than 15%, 10%, or 5%.

In some embodiments, the reference value is the GlcSph level in a test sample of the subject prior to the subject receiving treatment. In some embodiments, the reference value is measured in a reference sample obtained from a reference subject or a population of reference subjects. In certain embodiments, the reference subject or population of reference subjects is a healthy control. In particular embodiments, the reference subject or population of reference subjects does not have FTD or a decreased level of PGRN.

In some embodiments, step (a) of the methods described herein further comprises measuring the abundance of one or more bis(monoacylglycero)phosphate (BMP) species. In certain embodiments, the one or more BMP species comprise BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1e_20:4), BMP(20:4_22:6), BMP(18:0e_20:4), BMP(18:2_20:4), BMP(18:1_22:6), BMP(18:1_20:4), BMP(18:0_22:6), and/or BMP(18:3_22:5).

In some embodiments, the test sample or one or more reference values comprise or relate to whole blood, plasma, a cell, a tissue, serum, cerebrospinal fluid, interstitial fluid, sputum, urine, lymph, or a combination thereof. In particular embodiments, the test sample or one or more reference values comprise or relate to plasma. In certain embodiments, the cell is a blood cell, a brain cell, a peripheral blood mononuclear cell (PBMC), a bone marrow-derived macrophage (BMDM), a retinal pigmented epithelial (RPE) cell, an erythrocyte, a leukocyte, a neural cell, a microglial cell, a cerebral cortex cell, a spinal cord cell, a bone marrow cell, a liver cell, a kidney cell, a splenic cell, a skin cell, a fibroblast, a heart cell, a lymph node cell, or a combination thereof. In certain embodiments, the tissue comprises a lymph node, bone marrow, skin tissue, blood vessel tissue, lung tissue, spleen tissue, valvular tissue, or a combination thereof.

In some embodiments, the test sample comprises an endosome, a lysosome, an extracellular vesicle, an exosome, a microvesicle, or a combination thereof.

In some embodiments, the abundance of the GlcSph is measured using liquid chromatography-mass spectrometry (LC-MS), liquid chromatography-tandem mass spectrometry (LC-MS/MS), gas chromatography-mass spectrometry (GC-MS), gas chromatography-tandem mass spectrometry (GC-MS/MS), enzyme-linked immunosorbent assay (ELISA), or a combination thereof.

In some embodiments, an internal GlcSph standard is used when measuring the abundance of the GlcSph. In certain embodiments, the internal GlcSph standard comprises a GlcSph species that is not naturally present in the subject. In particular embodiments, the internal GlcSph standard comprises a deuterium-labeled GlcSph.

In some embodiments, the subject has one or more mutations in the granulin (GRN) gene. In some embodiments, the FTD is related to PGRN expression, processing, glycosylation, cellular uptake, trafficking, and/or function. In some embodiments, the FTD is associated with a decreased PGRN level.

In some embodiments, the subject and/or the reference subject is a human or a non-human primate. In some embodiments, the subject is a PGRN knockout mouse or PGRN knockout rat.

In another aspect, the present disclosure provides a kit for testing a compound or a dosing regimen thereof for treating a FTD in a subject, the kit comprising a GlcSph standard for measuring the abundance of GlcSph in a test sample from the subject.

In some embodiments of this aspect, the GlcSph standard comprises a GlcSph species that is not naturally present in the subject. In particular embodiments, the GlcSph standard comprises a deuterium-labeled GlcSph.

In some embodiments, the kit further comprises reagents for obtaining the test sample from the subject, processing the test sample, measuring the abundance of the GlcSph, or a combination thereof. In some embodiments, the kit further comprises instructions for use.

### BRIEF DESCRIPTION OF THE DRAWINGS

FIG. 1 shows a significant increase of GlcSph in plasma samples from GRN-linked FTD patients, but not in non-GRN FTD patients, compared to clinically normal controls who do not carry any genetic mutations.

FIG. 2 shows that following treatment with a PGRN derivative (Fusion 1), the GlcSph level in the plasma of Grn KO mice was reduced significantly compared to the GlcSph level in Grn KO mice not receiving the PGRN derivative.

FIG. 3A shows that after 6 weeks of dosing, Fusion 1 rescued GlcSph level in the brain of Grn KO/hTfR.KI mice, while Fusion 2 only showed a partial effect. Dose used was a weekly injection at 5 mpk for 6 weeks. Statistical analysis was a One-Way ANOVA with Dunnett's multiple comparison test (n=7-10 mice). **, P<0.01; ****, P<0.0001.

FIG. 3B shows that after 6 weeks of dosing, both Fusion 1 and Fusion 2 fully rescued GlcSph level in the liver of Grn KO/hTfR.KI mice. Dose used was a weekly injection at 5 mpk for 6 weeks. Statistical analysis was a One-Way ANOVA with Dunnett's multiple comparison test (n=7-10 mice). **, P<0.01; ****, P<0.0001.

FIG. 3C shows that after 6 weeks of dosing, both Fusion 1 and Fusion 2 rescued GlcSph level in the plasma of Grn KO/hTfR.KI mice. Dose used was a weekly injection at 5 mpk for 6 weeks. Statistical analysis was a One-Way ANOVA with Dunnett's multiple comparison test (n=7-10 mice). **, P<0.01; ****, P<0.0001.

### DETAILED DESCRIPTION

### I. Introduction

Loss of function mutations in the GRN gene have been linked to common forms of frontotemporal dementia (FTD). GRN encodes the soluble protein progranulin (PGRN), which has been implicated in neurite outgrowth, neuronal survival, lysosomal function, and inflammation. As therapeutic approaches are being developed for GRN-linked FTD, primarily focused on enhancing brain levels of PGRN, it is essential to identify disease-associated biomarkers in bodily fluids, such as plasma and CSF, to facilitate the discovery of candidate therapeutics. Within FTD cases, it is also important to investigate biomarkers that are either generally associated with FTD or specific for GRN-linked FTD. Importantly, biomarker discovery can also inform on disease mechanisms by revealing novel biology and biochemical pathways related to PGRN and/or its loss of function.

We conducted targeted lipidomics/metabolomics analysis by LC-MS and found a significant increase of glucosylsphingosine (GlcSph) in biological samples (e.g., plasma samples) from GRN-linked FTD patients (Δ25%, p<0.01), but not in sporadic FTD patients, compared to clinically normal controls who do not carry any genetic mutations, indicating GlcSph is a disease biomarker pertinent to GRN-linked FTD and a pathway biomarker for PGRN functional rescue. Our results show that a reduction of GlcSph level by about 20% would normalize the increased GlcSph level found in the sample of GRN-linked FTD patients relative to clinically normal controls.

### II. Definitions

As used herein, the singular forms “a,” “an,” and “the” include plural referents unless the content clearly dictates otherwise. Thus, for example, reference to “a cell” may include two or more such cells, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

The term “abundance” refers to the amount or concentration of a molecule, compound, or agent (e.g., GlcSph). The term includes an absolute amount or concentration, as well as a relative amount or concentration. In some embodiments, a reference standard (e.g., an internal GlcSph standard) is used for calibration in order to determine the absolute amount or concentration of the molecule, compound, or agent that is present (e.g., in a sample) and/or normalize to a control in order to determine a relative amount or concentration of the molecule, compound, or agent that is present.

The term “glucosylsphingosine” or “GlcSph” refers to a lysoglycosphingolipid having the structure depicted in Formula I:

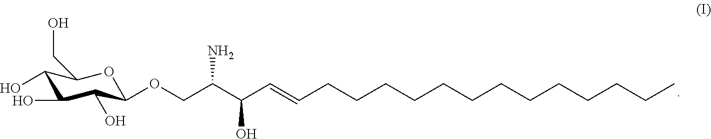

The term “bis(monoacylglycero)phosphate” or “BMP” refers to a glycerophospholipid that is negatively charged (e.g. at the pH normally present within late endosomes and lysosomes) having the structure depicted in Formula II:

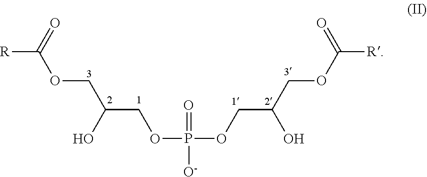

BMP molecules comprise two fatty acid side chains. R and R′ in Formula II represent independently selected saturated or unsaturated aliphatic chains, each of which typically contains 14, 16, 18, 20, or 22 carbon atoms. When a fatty acid side chain is unsaturated, it can contain 1, 2, 3, 4, 5, 6, or more carbon-carbon double bonds. Furthermore, a BMP molecule can contain one or two alkyl ether substituents, wherein the carbonyl oxygen of one or both fatty acid side chains is replaced with two hydrogen atoms. Nomenclature that is used herein to describe a particular BMP species refers to a species having two fatty acid side-chains, wherein the structures of the fatty acid side chains are indicated within parentheses in the BMP format (e.g., BMP(18:1_18:1)). The numerals follow the standard fatty acid notation format of number of “fatty acid carbon atoms:number of double bonds.” An “e-” prefix is used to indicate the presence of an alkyl ether substituent wherein the carbonyl oxygen of the fatty acid side chain is replaced with two hydrogen atoms. For example, the “e” in “BMP(16:0e_18:0)” denotes that the side chain having 16 carbon atoms is an alkyl ether substituent.

The term “PGRN level” refers to the amount, concentration, and/or activity level of progranulin that is present, either in a subject or in a sample (e.g., a sample obtained from a subject). A PGRN level can refer to an absolute amount, concentration, and/or activity level of PGRN that is present, or can refer to a relative amount, concentration, and/or activity level. The term also refers to the amount or concentration of a PGRN polypeptide and/or PGRN mRNA (e.g., expressed from a GRN gene) that is present.

The term “progranulin” or “PGRN” (also known as “proepithelin” or “acrogranin”) refers to a cysteine-rich protein encoded by the gene GRN, which maps to human chromosome 17q21. PGRN is a lysosomal protein as well as a secreted protein consisting of seven and a half tandem repeats of conserved granulin peptides, each of which is about 60 amino acid long and can be released through cleavage by various extracellular proteases (e.g., elastase) and lysosomal proteases (e.g., cathepsin L) (Kao et al., Nat Rev Neurosci. 18(6):325-333, 2017). Generally, PGRN is believed to play both cell-autonomous and non-cell autonomous roles in the control of innate immunity as well as the function of lysosomes, where it regulates the activity and levels of various cathepsins and other hydrolases (Kao et al., supra). PGRN also has a neurotrophic function and promotes neurite outgrowth and neuronal survival (Kao et al., supra). A PGRN polypeptide may comprise a human PGRN sequence. A PGRN polypeptide may be a pre-mature PGRN, e.g., in which the first 17 amino acids make up the signal peptide. A PGRN polypeptide may also be a mature PGRN, e.g., in which the 17-amino acid signal peptide is cleaved. As other non-limiting examples, a PGRN polypeptide may comprise a sequence from a non-human species, such as mouse (NCBI reference number NP_032201.2), rat (NCBI reference number NP_058809.2 or NP_001139314.1), or chimpanzee (NCBI reference number XP_016787144.1 or XP_016787145.1) in either pre-mature or mature form.

The term “progranulin derivative” or “PGRN derivative” refers to a modified PGRN. The modifications can be made to increase the druggability of PGRN, such as modifications that target PGRN to specific areas in the body, and/or improve its pharmacokinetic or phamacodynamic properties. Other modifications can be made to assist in its manufacture and/or shelf life. PGRN derivatives can include Fc-fusion proteins comprising PGRN attached to dimers of Fc polypeptides.

The term “bone marrow-derived macrophage” or “BMDM” refers to a macrophage cell that is generated or derived in vitro from a mammalian bone marrow (e.g., a bone marrow obtained from a subject). As a non-limiting example, BMDMs can be generated by culturing undifferentiated bone marrow cells in the presence of a cytokine such as macrophage colony-stimulating factor (M-CSF).

The terms “treatment,” “treating,” and the like are used herein to generally mean obtaining a desired pharmacologic and/or physiologic effect. “Treating” or “treatment” may refer to any indicia of success in the treatment or amelioration of frontotemporal dementia (FTD), including any objective or subjective parameter such as abatement, remission, improvement in patient survival, increase in survival time or rate, diminishing of symptoms or making the disorder more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “subject,” “individual,” and “patient,” as used interchangeably herein, refer to a mammal, including but not limited to humans, non-human primates, rodents (e.g., rats, mice, and guinea pigs), rabbits, dogs, cows, pigs, horses, and other mammalian species. In one embodiment, the subject is a human.

As used herein, a “therapeutic amount” or “therapeutically effective amount” of an agent is an amount of the agent that treats symptoms of a disease in a subject.

The term “administer” refers to a method of delivering agents, compounds, or compositions to the desired site of biological action. These methods include, but are not limited to, topical delivery, parenteral delivery, intravenous delivery, intradermal delivery, intramuscular delivery, subcutaneous delivery, intrathecal delivery, oral delivery, colonic delivery, rectal delivery, or intraperitoneal delivery.

### III. Methods of the Disclosure

Frontotemporal Dementia (FTD)

Frontotemporal dementia (FTD) is a progressive neurodegenerative disorder. FTD includes a spectrum of clinically, pathologically, and genetically heterogeneous diseases presenting selective involvement of the frontal and temporal lobes (Gazzina et al., Eur J Pharmacol. 817:76-85, 2017). Clinical manifestations of FTD include alterations in behavior and personality, frontal executive deficits, and language dysfunction. Based on the diversity of clinical phenotypes, different presentations have been identified, such as behavioral variants of FTD (bvFTD) and primary progressive aphasia (PPA), which can either be the nonfluent/agrammatic variant PPA (avPPA) or the semantic variant PPA (svPPA). These clinical presentations can also overlap with atypical parkinsonism, such as corticobasal syndrome (CBS), progressive supranuclear palsy (PSP), and amyotrophic lateral sclerosis (ALS) (Gazzina et al., supra). FTD is associated with various neuropathological hallmarks, including tau pathology in neurons and astrocytes or cytoplasmic ubiquitin inclusions in neurons. The Trans-activating DNA-binding Protein with a molecular weight of 43 kDa (TDP-43) is the most prominent, ubiquitinated protein pathology accumulating in the majority of cases of FTD as well as in ALS (Petkau and Leavitt, Trends Neurosci. 37(7):388-98, 2014). FTD is a significant cause of early-onset dementia with up to 80% of cases presenting between ages 45 and 64. The disease also presents a significant familial component, with about 30-50% of cases reporting family history of the disease (Petkau and Leavitt, supra).

FTD occurs both in familial and sporadic forms. Some forms of familial FTD have no known cause, while others are caused by genetic mutations. Mutations in numerous genes have been associated with FTD. Mutations in genes such as GRN, MAPT, and C9ORF72 are the most common causes of genetic FTD. Rare mutations have been identified in other genes such as VCP, TARDBP, CHMP2B, SQSTM1, UBQLN1, UBQLN2, TBK1, and CHCHD10. Mutations in neurodegenerative disease genes not commonly linked to FTD such as PSEN1, PSEN2, CTSF, CYP27A1 have also been identified in FTD subjects (Blauwendraat et al., Genetics in Medicine 20:240-249, 2018).

Glucosylsphingosine (GlcSph)

Provided herein are methods for identifying a subject having, or at risk of having, frontotemporal dementia (FTD), and/or for evaluating a compound or monitoring a subject's response to a compound, pharmaceutical composition, or dosing regimen thereof for treating FTD, in which the methods comprise measuring the abundance of glucosylsphingosine (GlcSph) in a test sample from the subject.

GlcSph is a substrate of glucocerebrosidase (GCase) and is found to accumulate in cells and tissues of human Gaucher disease patients and mouse models that exhibit reduced GCase activity. The accumulation of GlcSph is implicated in the visceral and neuronal pathologies observed in Gaucher disease.

In some embodiments, the abundance of GlcSph can be compared to a reference value. In some embodiments, a subject having, or at risk of having, FTD has an increased GlcSph level compared to the reference value, e.g., the abundance of the GlcSph in the test sample of the subject can be at least about 1.2-fold to about 5-fold or at least about 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, or 10-fold of the reference value. In some embodiments, the reference value is the GlcSph level in a test sample of the subject having, or at risk of having, FTD prior to the subject receiving treatment.

In some embodiments of the methods of the present disclosure, the reference value is measured in a reference sample obtained from a reference subject or a population of reference subjects. The reference subject or population of reference subjects can be a healthy control subject or a population of healthy control subjects. The reference subject or population of reference subjects can be a subject or a population of subjects who does not have FTD or a decreased level of PGRN. In some embodiments, after the subject having, or at risk of having, FTD receives treatment, the GlcSph level in a test sample from the subject can improve over the GlcSph level in a test sample from the subject prior to the subject receiving any treatment. In some embodiments, the improved GlcSph level is closer to the reference value (e.g., the reference value measured in a reference sample obtained from a healthy control subject or a population of healthy control subjects) than the GlcSph level in the subject having, or at risk of having, FTD prior to the subject receiving treatment, for example, the improved GlcSph level has a difference compared to the reference value of less than 15%, 10%, or 5%.

In some cases, in subjects having, or at risk of having, FTD, the increased GlcSph level compared to a reference value can be found in, e.g., whole blood, plasma, a cell, a tissue, serum, cerebrospinal fluid, interstitial fluid, sputum, urine, lymph, or a combination thereof of the subject. In particular embodiments, the increased GlcSph level can be found in the plasma of the subject. In some embodiments of the methods of the disclosure, the test sample taken from the subject having, or at risk of having, FTD or one or more reference values can comprise or relate to plasma.

Further, in subjects having, or at risk of having, FTD, the increased GlcSph level compared to a reference value can be found in the brain of the subject, for example, in the frontal lobe and/or temporal lobe of the brain. In particular embodiments, the increased GlcSph level can be found in one or more regions of the frontal lobe, e.g., superior frontal gyms, middle frontal gyms, inferior frontal gyms, and/or precentral gyms.

The test sample taken from the subject having, or at risk of having, FTD used in the methods described herein can comprise a cell, such as a blood cell, a brain cell, a peripheral blood mononuclear cell (PBMC), a bone marrow-derived macrophage (BMDM), a retinal pigmented epithelial (RPE) cell, an erythrocyte, a leukocyte, a neural cell, a microglial cell, a cerebral cortex cell, a spinal cord cell, a bone marrow cell, a liver cell, a kidney cell, a splenic cell, a lung cell, an eye cell, a chorionic villus cell, a muscle cell, a skin cell, a fibroblast, a heart cell, a lymph node cell, or a combination thereof. In some embodiments, the test sample comprises a blood cell. In some embodiments, the test sample comprises a brain cell.

The test sample taken from the subject having, or at risk of having, FTD used in the methods described herein can comprise a tissue, such as brain tissue, cerebral cortex tissue, spinal cord tissue, liver tissue, kidney tissue, muscle tissue, heart tissue, eye tissue, retinal tissue, a lymph node, bone marrow, skin tissue, blood vessel tissue, lung tissue, spleen tissue, valvular tissue, or a combination thereof. In some embodiments, the test sample comprises brain tissue, such as brain tissue from the frontal lobe or temporal lobe of the subject's brain. In particular embodiments, the brain tissue used in the test sample can be from the superior frontal gyms, middle frontal gyms, inferior frontal gyms, and/or precentral gyms.

The test sample taken from the subject having, or at risk of having, FTD used in the methods described herein can comprise an endosome, a lysosome, an extracellular vesicle, an exosome, a microvesicle, or a combination thereof.

In some embodiments, an internal GlcSph standard is used to measure the abundance of GlcSph in a test sample from a subject having, or at risk of having, FTD and/or determine a reference value (e.g., measure the abundance of GlcSph in a reference sample). For example, a known amount of the internal GlcSph standard can be added to a sample (e.g., a test sample and/or a reference sample) to serve as a calibration point such that the amount of GlcSph that is present in the sample can be determined. In some embodiments, a reagent used in the extraction or isolation of GlcSph from a sample (e.g., methanol) is “spiked” with the internal GlcSph standard. Typically, the internal GlcSph standard is one that does not naturally occur in the subject. In some embodiments, the internal GlcSph is a deuterium-labeled GlcSph, such as GlcSph(d5) used in the Examples.

Identification of Subjects Having, or at Risk of Having, FTD Using GlcSph

In some embodiments, a subject is determined to have FTD or a decreased level of PGRN when the abundance of GlcSph in a test sample from the subject is higher than a reference value. The reference value can be measured in a reference sample obtained from a reference subject or a population of reference subjects, e.g., a healthy control or subjects who do not have FTD or a decreased level of PGRN. In further embodiments, a subject is determined to have FTD or a decreased level of PGRN when the subject has one or more mutations in the granulin (GRN) gene. In some embodiments, the test sample from the subject can be a plasma sample.

In some embodiments, a subject is determined to have FTD or a decreased level of PGRN when the abundance of GlcSph (e.g., measured in a test sample) is at least about 1.2-fold (e.g., about 1.2-fold, 1.3-fold, 1.4-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or more) of a reference value, e.g., a reference value measured in a reference sample obtained from a reference subject or a population of reference subjects.

In some embodiments, a subject is determined to have FTD or a decreased level of PGRN when the abundance of GlcSph (e.g., measured in a test sample) is at least about 1.2-fold to about 5-fold (e.g., at least about 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, or 5-fold) of a reference value, e.g., a reference value measured in a reference sample obtained from a reference subject or a population of reference subjects. In some embodiments, a subject is determined to have FTD or a decreased level of PGRN when the abundance of GlcSph is about 2-fold to about 3-fold (e.g., about 2-fold, 2.1-fold, 2.2-fold, 2.3-fold, 2.4-fold, 2.5-fold, 2.6-fold, 2.7-fold, 2.8-fold, 2.9-fold, or 3-fold) of a reference value, e.g., a reference value measured in a reference sample obtained from a reference subject or a population of reference subjects.

Bis(monoacylglycero)phosphate (BMP)

In some embodiments, in addition to measuring the abundance of GlcSph in methods for identifying a subject having, or at risk of having, FTD, and/or for evaluating a compound or monitoring a subject's response to a compound, pharmaceutical composition, or dosing regimen thereof for treating FTD as described herein, the methods further comprise measuring the abundance of one or more bis(monoacylglycero)phosphate (BMP) species in a test sample from the subject. In some embodiments, the methods provided herein further comprise administering to the subject a compound for improving the one or more BMP species levels for treating FTD.

In some embodiments of the methods, both the abundance of GlcSph and the abundance of one or more BMP species can be measured from the same test sample from the subject. In other embodiments, two test samples (e.g., two test samples taken at the same time) can be taken from the subject, in which one test sample can be used to measure the abundance of GlcSph, while the other test sample can be used to measure the abundance of one or more BMP species. The two test samples can be taken from the same fluid, cell, or tissue of the subject (e.g., whole blood, plasma, a cell, a tissue, serum, cerebrospinal fluid, interstitial fluid, sputum, urine, or lymph). In other embodiments, the two test samples can be taken from different fluids, cells, or tissues of the subject, e.g., one sample can be plasma, while the other sample can be brain tissue.

In some embodiments, in addition to measuring the abundance of GlcSph, the abundance of a single BMP species is measured. In some embodiments, the abundance of two or more BMP species is measured. In some embodiments, the abundance of at least two, three, four, five, or more of the BMP species in Table 1 is measured. When the abundance of two or more BMP species is measured, any combination of different BMP species can be used.

TABLE 1

| | Total carbon |
|---|---|
| | atoms:total |
| Name | unsaturations |
| BMP(14:0_14:0) | BMP(28:0) |
| BMP(14:0_16:0) | BMP(30:0) |
| BMP(14:0_16:1) | BMP(30:1) |
| BMP(14:0_18:0) | BMP(32:0) |
| BMP(14:0_18:1) | BMP(32:1) |
| BMP(14:0_18:2) | BMP(32:2) |
| BMP(14:0_18:3) | BMP(32:3) |
| BMP(14:0_20:1) | BMP(34:1) |
| BMP(14:0_20:2) | BMP(34:2) |
| BMP(14:0_20:3) | BMP(34:3) |
| BMP(14:0_20:4) | BMP(34:4) |
| BMP(14:0_20:5) | BMP(34:5) |
| BMP(14:0_22:4) | BMP(36:4) |
| BMP(14:0_22:5) | BMP(36:5) |
| BMP(14:0_22:6) | BMP(36:6) |
| BMP(16:0_16:0) | BMP(32:0) |
| BMP(16:0_16:1) | BMP(32:1) |
| BMP(16:0_18:0) | BMP(34:0) |
| BMP(16:0_18:1) | BMP(34:1) |
| BMP(16:0_18:2) | BMP(34:2) |
| BMP(16:0_18:3) | BMP(34:3) |
| BMP(16:0_20:1) | BMP(36:1) |
| BMP(16:0_20:2) | BMP(36:2) |
| BMP(16:0_20:3) | BMP(36:3) |
| BMP(16:0_20:4) | BMP(36:4) |
| BMP(16:0_20:5) | BMP(36:5) |
| BMP(16:0_22:4) | BMP(38:4) |
| BMP(16:0_22:5) | BMP(38:5) |
| BMP(16:0_22:6) | BMP(38:6) |
| BMP(16:1_16:1) | BMP(32:2) |
| BMP(16:1_18:0) | BMP(34:1) |
| BMP(16:1_18:1) | BMP(34:2) |
| BMP(16:1_18:2) | BMP(34:3) |
| BMP(16:1_18:3) | BMP(34:4) |
| BMP(16:1_20:1) | BMP(36:2) |
| BMP(16:1_20:2) | BMP(36:3) |
| BMP(16:1_20:3) | BMP(36:4) |
| BMP(16:1_20:4) | BMP(36:5) |
| BMP(16:1_20:5) | BMP(36:6) |
| BMP(16:1_22:4) | BMP(38:5) |
| BMP(16:1_22:5) | BMP(38:6) |
| BMP(16:1_22:6) | BMP(38:7) |
| BMP(16:0e_14:0) | BMP(40:0) |
| BMP(16:0e_16:0) | BMP(32:0) |
| BMP(16:0e_18:0) | BMP(34:0) |
| BMP(16:0e_18:1) | BMP(34:1) |
| BMP(16:0e_18:2) | BMP(34:2) |
| BMP(16:0e_18:3) | BMP(34:3) |
| BMP(16:0e_20:3) | BMP(36:3) |
| BMP(16:0e_20:4) | BMP(36:4) |
| BMP(16:0e_20:5) | BMP(36:5) |
| BMP(16:0e_22:4) | BMP(38:4) |
| BMP(16:0e_22:6) | BMP(38:6) |
| BMP(16:1e_14:0) | BMP(30:1) |
| BMP(16:1e_16:0) | BMP(32:1) |
| BMP(16:1e_18:0) | BMP(34:1) |
| BMP(16:1e_18:1) | BMP(34:2) |
| BMP(16:1e_18:2) | BMP(34:3) |
| BMP(16:1e_18:3) | BMP(34:4) |
| BMP(16:1e_20:3) | BMP(36:4) |
| BMP(16:1e_20:4) | BMP(36:5) |
| BMP(16:1e_20:5) | BMP(36:6) |
| BMP(16:1e_22:4) | BMP(38:5) |
| BMP(16:1e_22:6) | BMP(38:7) |
| BMP(18:0_18:0) | BMP(36:0) |
| BMP(18:0_18:1) | BMP(36:1) |
| BMP(18:0_18:2) | BMP(36:2) |
| BMP(18:0_18:3) | BMP(36:3) |
| BMP(18:0_20:1) | BMP(38:1) |
| BMP(18:0_20:2) | BMP(38:2) |
| BMP(18:0_20:3) | BMP(38:3) |
| BMP(18:0_20:4) | BMP(38:4) |
| BMP(18:0_20:5) | BMP(38:5) |
| BMP(18:0_22:4) | BMP(40:4) |
| BMP(18:0_22:5) | BMP(40:5) |
| BMP(18:0_22:6) | BMP(40:6) |
| BMP(18:1_18:1) | BMP(36:2) |
| BMP(18:1_18:2) | BMP(36:3) |
| BMP(18:1_18:3) | BMP(36:4) |
| BMP(18:1_20:1) | BMP(38:2) |
| BMP(18:1_20:2) | BMP(38:3) |
| BMP(18:1_20:3) | BMP(38:4) |
| BMP(18:1_20:4) | BMP(38:5) |
| BMP(18:1_20:5) | BMP(38:6) |
| BMP(18:1_22:4) | BMP(40:5) |
| BMP(18:1_22:5) | BMP(40:6) |
| BMP(18:1_22:6) | BMP(40:7) |
| BMP(18:2_18:2) | BMP(36:4) |
| BMP(18:2_18:3) | BMP(36:5) |
| BMP(18:2_20:1) | BMP(38:3) |
| BMP(18:2_20:2) | BMP(38:4) |
| BMP(18:2_20:3) | BMP(38:5) |
| BMP(18:2_20:4) | BMP(38:6) |
| BMP(18:2_20:5) | BMP(38:7) |
| BMP(18:2_22:4) | BMP(40:6) |
| BMP(18:2_22:5) | BMP(40:7) |
| BMP(18:2_22:6) | BMP(40:8) |
| BMP(18:3_18:3) | BMP(36:6) |
| BMP(18:3_20:1) | BMP(38:4) |
| BMP(18:3_20:2) | BMP(38:5) |
| BMP(18:3_20:3) | BMP(38:6) |
| BMP(18:3_20:4) | BMP(38:7) |
| BMP(18:3_20:5) | BMP(38:8) |
| BMP(18:3_22:4) | BMP(40:7) |
| BMP(18:3_22:5) | BMP(40:8) |
| BMP(18:3_22:6) | BMP(40:9) |
| BMP(18:0e_14:0) | BMP(32:0) |
| BMP(18:0e_16:0) | BMP(34:0) |
| BMP(18:0e_18:0) | BMP(36:0) |
| BMP(18:0e_18:1) | BMP(36:1) |
| BMP(18:0e_18:2) | BMP(36:2) |
| BMP(18:0e_18:3) | BMP(36:3) |
| BMP(18:0e_20:3) | BMP(38:3) |
| BMP(18:0e_20:4) | BMP(38:4) |
| BMP(18:0e_20:5) | BMP(38:5) |
| BMP(18:0e_22:4) | BMP(40:4) |
| BMP(18:0e_22:6) | BMP(40:6) |
| BMP(18:1e_14:0) | BMP(32:1) |
| BMP(18:1e_16:0) | BMP(34:1) |
| BMP(18:1e_18:0) | BMP(36:1) |
| BMP(18:1e_18:1) | BMP(36:2) |
| BMP(18:1e_18:2) | BMP(36:3) |
| BMP(18:1e_18:3) | BMP(36:4) |
| BMP(18:1e_20:3) | BMP(38:4) |
| BMP(18:1e_20:4) | BMP(38:5) |
| BMP(18:1e_20:5) | BMP(38:6) |
| BMP(18:1e_22:4) | BMP(40:5) |
| BMP(18:1e_22:6) | BMP(40:7) |
| BMP(20:3_20:3) | BMP(40:6) |
| BMP(20:3_20:4) | BMP(40:7) |
| BMP(20:3_20:5) | BMP(40:8) |
| BMP(20:3_22:4) | BMP(42:7) |
| BMP(20:3_22:5) | BMP(42:8) |
| BMP(20:3_22:6) | BMP(42:9) |
| BMP(20:4_20:4) | BMP(40:8) |
| BMP(20:4_20:5) | BMP(40:9) |
| BMP(20:4_22:4) | BMP(42:8) |
| BMP(20:4_22:5) | BMP(42:9) |
| BMP(20:4_22:6) | BMP(42:10) |
| BMP(20:5_20:5) | BMP(40:10) |
| BMP(20:5_22:4) | BMP(42:9) |
| BMP(20:5_22:5) | BMP(42:10) |
| BMP(20:5_22:6) | BMP(42:11) |
| BMP(22:4_22:4) | BMP(44:8) |
| BMP(22:4_22:5) | BMP(44:9) |
| BMP(22:4_22:6) | BMP(44:10) |
| BMP(22:6_22:6) | BMP(44:12) |

In some embodiments, the one or more BMP species comprise two or more BMP species. In some embodiments, the one or more BMP species comprise BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1e_20:4), BMP(18:3_22:5), BMP(20:4_22:6), BMP(18:0e_20:4), BMP(18:2_20:4), BMP(18:1_22:6), BMP(18:1_20:4), BMP(18:0_22:6), or a combination thereof.

In some embodiments, the one or more BMP species comprise BMP(18:1_18:1), BMP(18:0_20:4), BMP(20:4_20:4), BMP(22:6_22:6), BMP(20:4_22:6), BMP(18:1_22:6), BMP(18:1_20:4), BMP(18:0_22:6), BMP(18:3_22:5), or a combination thereof.

In some embodiments, the test sample comprises a cultured cell and the one or more BMP species comprise BMP(18:1_18:1). In some embodiments, the test sample comprises plasma, tissue, urine, cerebrospinal fluid (CSF), and/or brain or liver tissue, and the one or more BMP species comprise BMP(22:6_22:6). In some embodiments, the test sample comprises liver tissue and the one or more BMP species comprise BMP(22:6_22:6), BMP(18:3_22:5), or a combination thereof. In some embodiments, the test sample comprises CSF or urine and the one or more BMP species comprise BMP(22:6_22:6). In some embodiments, the test sample comprises microglia and the one or more BMP species comprise BMP(18:3_22:5).

In some embodiments, the abundance of more than one BMP species can be summed, and the total abundance will be compared to a reference value. For example, the abundance of each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, or more BMP species (e.g., the BMP species listed in Table 1) can be summed, and the total abundance then compared to a reference value.

In some cases, one or more BMP species may be differentially expressed (e.g., more or less abundant) in one type of sample when compared to another, such as, for example, cell-based samples (e.g., cultured cells) versus tissue-based or blood samples. Accordingly, in some embodiments, the selection of the one or more BMP species (i.e., for the measurement of abundance) depends on the type of sample. In some embodiments, the one or more BMP species comprise BMP(18:1_18:1), e.g., when a sample (e.g., a test sample and/or a reference sample) is bone marrow-derived macrophage (BMDM). In other embodiments, the one or more BMP species comprise BMP(22:6_22:6), e.g., when a sample comprises tissue (e.g., brain tissue, liver tissue) or plasma, urine, or CSF.

In some embodiments, an internal BMP standard (e.g., BMP(14:0_14:0)) is used to measure the abundance of one or more BMP species in a sample and/or determine a reference value (e.g., measure the abundance of one or more BMP species in a reference sample). For example, a known amount of the internal BMP standard can be added to a sample (e.g., a test sample and/or a reference sample) to serve as a calibration point such that the amount of one or more BMP species that are present in the sample can be determined. In some embodiments, a reagent used in the extraction or isolation of BMP from a sample (e.g., methanol) is “spiked” with the internal BMP standard. Typically, the internal BMP standard will be one that does not naturally occur in the subject.

Identification of Subjects Having, or at Risk of Having, FTD Using BMP Species

In some embodiments of the methods described herein, in addition to using the abundance of GlcSph to identify subjects having, or at risk of having, FTD, the abundance of one or more BMP species can also be used to identify such subjects. A subject is determined to have FTD or a decreased level of PGRN when the abundance of at least one (e.g., a at least 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, or more) BMP species (e.g., the BMP species listed in Table 1) in a test sample is higher when the test sample is a BMDM or lower when the test sample is liver, brain, cerebrospinal fluid, plasma, or urine than a reference value measured in a corresponding cell, tissue, or fluid reference sample obtained from a reference subject or a population of reference subjects.

In some embodiments, a subject is determined to have FTD or a decreased level of PGRN when the abundance of at least one (e.g., 1, 2, 3, 4, 5, 6, 7, 8, 9, 10, 11, 12, 13, 14, 15, 16, 17, 18, 19, 20, 21, 22, or all 23) of the BMP species selected from the group consisting of BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1e_20:4), BMP(20:4_22:6), BMP(18:0e_20:4), BMP(18:2_20:4), BMP(18:1_22:6), BMP(18:1_20:4), BMP(18:0_22:6), and BMP(18:3_22:5) is elevated in BMDM or decreased in liver, brain, cerebrospinal fluid, plasma, or urine compared to a reference value measured in a corresponding cell, tissue, or fluid reference sample obtained from a reference subject or a population of reference subjects.

In some embodiments, a subject is determined to have FTD or a decreased level of PGRN when the abundance of at least one (e.g., 1, 2, 3, 4, 5, 6, 7, or all 8) of the BMP species selected from the group consisting of BMP(18:1_18:1), BMP(18:0_20:4), BMP(20:4_20:4), BMP(22:6_22:6), BMP(20:4_2:6), BMP(18:1_22:6), BMP(18:1_20:4), BMP(18:0_22:6) and BMP(18:3_22:5) is elevated in BMDM or decreased in liver, brain, cerebrospinal fluid, plasma, or urine compared to a reference value measured in a corresponding cell, tissue, or fluid reference sample obtained from a reference subject or a population of reference subjects.

In some embodiments, a subject is determined to have FTD or a decreased level of PGRN when BMP(18:1_18:1) levels are elevated in BMDM compared to a reference value measured in a corresponding BMDM reference sample obtained from a reference subject or a population of reference subjects. In other embodiments, a subject is determined to have FTD or a decreased level of PGRN when BMP(22:6_22:6) are decreased in plasma, urine, cerebrospinal fluid (CSF), and/or brain or liver tissue compared to a reference value measured in a corresponding cell, tissue, or fluid reference sample obtained from a reference subject or a population of reference subjects. In other embodiments, a subject is determined to have FTD or a decreased level of PGRN when BMP(22:6_22:6) and/or BMP(18:3_22:5) levels are decreased in liver tissue. In other embodiments, a subject is determined to have FTD or a decreased level of PGRN when BMP(18:3_22:5) levels are decreased in microglia.

In some embodiments, a subject is determined to have FTD or a decreased level of PGRN when the abundance of at least one of the BMP species (e.g., measured in a test sample) is at least about 1.2-fold (e.g., about 1.2-fold, 1.3-fold, 1.4-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or more) higher in BMDM or lower in liver, brain, cerebrospinal fluid, plasma, or urine compared to a reference value measured in a corresponding cell, tissue, or fluid reference sample obtained from a reference subject or a population of reference subjects.

In some embodiments, a subject is determined to have FTD or a decreased level of PGRN when the abundance of at least one of the BMP species (e.g., measured in a test sample) is at least about 1.2-fold to about 5-fold (e.g., at least about 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, or 5-fold) of a reference value (e.g., a corresponding reference value measured in a corresponding cell, tissue, or fluid reference sample obtained from a reference subject or a population of reference subjects). In some embodiments, a subject is determined to have FTD or a decreased level of PGRN when the abundance of at least one of the BMP species is about 2-fold to about 3-fold (e.g., about 2-fold, 2.1-fold, 2.2-fold, 2.3-fold, 2.4-fold, 2.5-fold, 2.6-fold, 2.7-fold, 2.8-fold, 2.9-fold, or 3-fold) higher in BMDM or lower in liver, brain, cerebrospinal fluid, plasma, or urine compared to a reference value measured in a corresponding cell, tissue, or fluid reference sample obtained from a reference subject or a population of reference subjects.

Detection Techniques

In some embodiments, mass spectrometry (MS) is used to detect and/or measure the abundance of GlcSph and/or BMP according to methods of the present disclosure. Any suitable MS technique can be used. Such techniques include, but are not limited to, single mass spectrometry (MS) that uses a single mass analyzer (e.g., quadrupole) and tandem mass spectrometry (MS/MS) that uses a series of mass analyzers (e.g., three mass analyzers) to perform multiple rounds of mass spectrometry, typically having a molecule fragmentation step in between. MS and MS/MS techniques can be coupled with liquid chromatography (LC) (e.g., high performance liquid chromatography (HPLC) or ultra high performance liquid chromatography (UHPLC)) or gas chromatography (GC) techniques. Such liquid chromatography-mass spectrometry (LC-MS), liquid chromatography-tandem mass spectrometry (LC-MS/MS), gas chromatography-mass spectrometry (GC-MS), and gas chromatography-tandem mass spectrometry (GC-MS/MS) methods allow for enhanced mass resolving and mass determining over what is typically possible with MS or MS/MS alone.

In some embodiments, antibody-based methods are used to detect and/or measure the abundance of GlcSph and/or BMP. Non-limiting examples of suitable methods include enzyme-linked immunosorbent assay (ELISA), immunofluorescence, and radioimmunoassay (RIA) techniques. Methods for performing ELISA, immunofluorescence, and MA techniques are known in the art.

Any number of sample types can be used as a test sample and/or reference sample in methods of the present disclosure so long as the sample comprises GlcSph and/or BMP in an amount sufficient for detection such that the abundance can be measured. Non-limiting examples include blood (e.g., whole blood, plasma, serum), cells, tissues, fluids (e.g., cerebrospinal fluid, urine, bronchioalveolar lavage fluid, lymph, semen, breast milk, amniotic fluid), feces, sputum, or any combination thereof. Non-limiting examples of suitable cell types include blood cells (e.g., peripheral blood mononuclear cells (PBMCs), erythrocytes, leukocytes), neural cells (e.g., brain cells, cerebral cortex cells, spinal cord cells), bone marrow-derived macrophages (BMDMs), bone marrow cells, liver cells, kidney cells, splenic cells, lung cells, eye cells (e.g., retinal cells such as retinal pigmented epithelial (RPE) cells), chorionic villus cells, muscle cells, skin cells, fibroblasts, heart cells, lymph node cells, or a combination thereof. In some embodiments, the sample comprises a portion of a cell. In some embodiments, the sample is purified from a cell or a tissue. Non-limiting examples of purified samples include endosomes, lysosomes, extracellular vesicles (e.g., exosomes, microvesicles), and combinations thereof.

In some embodiments, the sample (e.g., test sample and/or reference sample) comprises plasma.

In some embodiments, the sample (e.g., test sample and/or reference sample) comprises a brain cell or tissue. The brain cell or tissue can be from the frontal lobe or temporal lobe of the brain. In particular embodiments, the brain cell or tissue can be from the superior frontal gyms, middle frontal gyms, inferior frontal gyms, or precentral gyms of the frontal lobe of the brain.

In some embodiments, the sample (e.g., test sample and/or reference sample) comprises a cell that is a cultured cell. Non-limiting examples include BMDMs and RPE cells. BMDMs can be obtained, for example, by procuring a sample comprising PBMCs and culturing the monocytes contained therein.

Non-limiting examples of suitable tissue sample types include neural tissue (e.g., brain tissue, cerebral cortex tissue, spinal cord tissue), liver tissue, kidney tissue, muscle tissue, heart tissue, eye tissue (e.g., retinal tissue), lymph nodes, bone marrow, skin tissue, blood vessel tissue, lung tissue, spleen tissue, valvular tissue, and a combination thereof. In some embodiments, a test sample and/or a reference sample comprises brain tissue or liver tissue.

Monitoring Response to Treatment

In one aspect, the present disclosure provides methods for monitoring PGRN levels in a subject. In another aspect, provided are methods for monitoring a subject's response to a compound, pharmaceutical composition, or dosing regimen thereof or response to any therapy or therapeutic for treating FTD.

Typically, the abundance of GlcSph in a test sample from a subject having, or at risk of having, FTD can be compared to one or more reference values (e.g., a corresponding reference value). In addition to the abundance of GlcSph, the abundance of one or more BMP species can also be measured in a test sample from a subject having, or at risk of having, FTD and compared to one or more reference values (e.g., a corresponding reference value). In some embodiments, a GlcSph value and/or a BMP value is measured before the subject receives treatment and at one or more later time points after the subject receives treatment. The abundance value taken at a later time point after treatment can be compared to the value prior to treatment to determine how the subject is responding to the therapy. The abundance value taken at a later time point can also be compared to a reference value, such as that of a healthy control, to determine how the subject is responding to the therapy. The reference value can be from cells, tissues, or fluids of a healthy control, corresponding to the cell, tissue, or fluid of the test sample of the subject.

In some embodiments, the reference value is the abundance of GlcSph that is measured in a reference sample. In some embodiments, the reference value is the abundance of one or more BMP species that is measured in a reference sample. The reference value can be a measured abundance value (e.g., abundance value measured in the reference sample), or can be derived or extrapolated from a measured abundance value. In some embodiments, the reference value is a range of values, e.g., when the reference values are obtained from a plurality of samples or a population of subjects. Furthermore, the reference value can be presented as a single value (e.g., a measured abundance value, a mean value, or a median value) or a range of values, with or without a standard deviation or standard of error.

When two or more test samples are obtained (e.g., from a subject), the time points at which they are obtained can be separated by about 1, 5, 10, 15, 20, 25, 30, 35, 40, 45, 50, 55, 60, or more minutes; about 1, 2, 3, 4, 5, 6, 7, 8, 9, 10, 11, 12, 13, 14, 15, 16, 17, 18, 19, 20, 21, 22, 23, 24, or more hours; about 1, 2, 3, 4, 5, 6, 7, or more days; about 1, 2, 3, 4, 5, 6, 7, 8, 9, 10, or more weeks; or even longer. When three or more test samples are obtained, the time intervals between when each test sample is obtained can all be the same, the intervals can all be different, or a combination thereof.

In some embodiments, the first test sample is obtained before the subject has been treated for FTD (i.e., a pre-treatment test sample) and the second test sample is obtained after the subject has been treated for FTD (i.e., a post-treatment test sample). In some embodiments, both the first test sample and the second test sample are obtained from a subject after the subject has been treated, i.e., the first test sample is obtained from the subject at an earlier time point during treatment than the second test sample. In some embodiments, more than one (e.g., 1, 2, 3, 4, 5, 6, 7, 8, 9, 10, or more) pre-treatment and/or post-treatment test samples are obtained from the subject. Furthermore, the number of pre-treatment and post-treatment test samples that are obtained need not be the same.

In some embodiments of the methods, both the abundance of GlcSph and the abundance of one or more BMP species can be measured from the same test sample from the subject. In other embodiments, two test samples (e.g., two test samples taken at the same time, or two test samples taken at different times) can be taken from the subject, in which one test sample can be used to measure the abundance of GlcSph, while the other test sample can be used to measure the abundance of one or more BMP species. The two test samples can be taken from the same fluid, cell, or tissue of the subject (e.g., whole blood, plasma, a cell, a tissue, serum, cerebrospinal fluid, interstitial fluid, sputum, urine, or lymph). In other embodiments, the two test samples can be taken from different fluids, cells, or tissues of the subject, e.g., one sample can be plasma, while the other sample can be brain cells or brain tissue.

In some embodiments, it may be determined that the subject is not responding to the treatment when the abundance of GlcSph measured is within about 1%, 2%, 3%, 4%, 5%, 6%, 7%, 8%, 9%, 10%, 11%, 12%, 13%, 14%, 15%, 16%, 17%, 18%, 19%, or 20% of the reference value taken in a reference sample from the subject before the subject receiving any treatment.

In some embodiments, it may be determined that the subject is responding to the treatment when the abundance of GlcSph measured is within about 1%, 2%, 3%, 4%, 5%, 6%, 7%, 8%, 9%, 10%, 11%, 12%, 13%, 14%, 15%, 16%, 17%, 18%, 19%, or 20% of the reference value taken in a reference sample from a healthy control subject.

In some embodiments, it may be determined that the subject is not responding to the treatment when the abundance of one or more BMP species measured is within about 1%, 2%, 3%, 4%, 5%, 6%, 7%, 8%, 9%, 10%, 11%, 12%, 13%, 14%, 15%, 16%, 17%, 18%, 19%, or 20% of the reference value taken in a reference sample from the subject before the subject receiving any treatment.

In some embodiments, it may be determined that the subject is responding to the treatment when the abundance of one or more BMP species measured is within about 1%, 2%, 3%, 4%, 5%, 6%, 7%, 8%, 9%, 10%, 11%, 12%, 13%, 14%, 15%, 16%, 17%, 18%, 19%, or 20% of the reference value taken in a reference sample from a healthy control subject.

In some embodiments, it may be determined that the subject is not responding to the treatment when the abundance of one or more BMP species measured is more than about 30%, 35%, 40%, 45%, or 50% higher or lower than the reference value.

When a subject is not responding to treatment for FTD, in some embodiments, the dosage of one or more therapeutic agents (e.g., PGRN) is altered (e.g., increased) and/or the dosing interval is altered (e.g., the time between doses is decreased). In some embodiments, when a subject is not responding to treatment, a different therapeutic agent is selected. In some embodiments, when a subject is not responding to treatment, one or more therapeutic agents is discontinued.

Compounds for Treating FTD

In some embodiments of the methods disclosed herein, a compound for treating FTD can be PGRN or a PGRN derivative. A PGRN derivative refers to a modified form of PGRN, which is modified to increase the druggability of PGRN, target PGRN to specific areas in the body, improve its pharmacokinetic or phamacodynamic properties, assist in its manufacture and/or shelf life.

PGRN derivatives can include Fc-fusion proteins comprising PGRN attached to dimers of Fc polypeptides. In such fusion proteins, in some embodiments, the fusion protein can contain one PGRN molecule and a dimer of Fc polypeptides. For example, the PGRN can be fused, directly or via a linker, to the N-terminus or C-terminus of an Fc polypeptide in the dimer. In other embodiments, the fusion protein can contain two PGRN molecules and a dimer of Fc polypeptides. For example, a first PGRN can be fused, directly or via a linker, to the N-terminus or C-terminus of the first Fc polypeptide in the dimer, and a second PGRN can be fused, directly or via a linker, to the N-terminus or C-terminus of the second Fc polypeptide in the dimer. Such fusion proteins are described in detail in International Patent Publication No. WO 2019/246071, the disclosure of which is hereby incorporated by reference in its entirety.

In some embodiments of the methods disclosed herein, a compound for treating FTD can be an expression construct comprising a transgene encoding wild-type PGRN in a gene therapy approach that can lead to increased PGRN levels in a subject having one or more mutations in the granulin (GRN) gene. For example, a recombinant adeno-associated viral vector comprising the expression construct can be used to deliver to the subject a wild-type GRN gene that does not have any mutations. Such gene therapies are described in, e.g., US Patent Publication No. US 2020/0071680, the disclosure of which is hereby incorporated by reference in its entirety.

In further embodiments, a compound for treating FTD can be a sortilin inhibitor, e.g., an anti-sortilin antibody. Sortilin is a type-I transmembrane protein that is a receptor for several ligands and also functions to sort select cargos from the trans-Golgi network to late endosomes and lysosomes for degradation. Sortilin binds to PGRN and targets it for lysosomal degradation, thus negatively regulating the level of PGRN. A sortilin inhibitor used for treating FTD refers to a compound that can inhibit the interaction between sortilin and PGRN, prevent sortilin from binding to PGRN, and/or decrease the level of sortilin. In some embodiments, a sortilin inhibitor can be an anti-sortilin antibody. Anti-sortilin antibodies and variants thereof are described in, e.g., International Patent Publication Nos. WO 2016/164637 and WO 2020/014617, the disclosures of which are hereby incorporated by reference in their entirety. Specific antibodies described in WO 2016/164637 include clones named S-2, S-5, S-6, S-8, S-14, S-15, S-15-10-7, S-15-6, S-18, S-19, S-20, S-21, S-22, S-29, S-30, S-45, S-49, S-51, S-57, S-60, S-60-1, S-60-2, S-60-3, S-60-4, S-60-5, S-60-6, S-60-7, S-60-8, S-60-9, S-61, S-63, S-64, S-65, S-72, S-82, and S-83. Specific anti-sortilin antibodies described in WO 2020/014617 include the clones named S-60, S-60-1, S-60-2, S-60-3, S-60-4, S-60-7, S-60-8, S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10[N33E], S-60-15.11 [N33W], S-60-[N33F], S-60-15.13 [N33I], S-60-15.14 [N33V], S-60-15.15 [N33A], S-60-15.16 [N33M], S-60-15.17 [N33L], S-60-16; S-60-18, S-60-19, S-60-24, and variants with heavy chain having Fc LALAPS with and without terminal lysine (e.g., S-60-15.1 [N33T]). In some embodiments, the anti-sortilin antibody is AL101 or AL100.

### IV. Kits

In another aspect, the present disclosure provides kits for use in monitoring PGRN levels in a subject (e.g., a test subject and/or a reference subject or population of reference subjects). In some embodiments, the kits are for use in measuring or calibrating the abundance of GlcSph (e.g., in a test sample obtained from a subject and/or a reference sample obtained from a reference subject or a population of reference subjects). In some embodiments, the kits comprise a GlcSph standard (e.g., an internal GlcSph standard) that can be used for measuring or calibrating the abundance of GlcSph (e.g., in a sample such as a test and/or reference sample). In some embodiments, the GlcSph standard comprises a GlcSph that is not naturally present in the subject. In some embodiments, the GlcSph standard comprises a GlcSph that is not naturally present in humans, non-human primates, rodents, dogs, and/or pigs. In some embodiments, the GlcSph standard comprises a GlcSph that is not naturally present in humans. In some embodiments, the GlcSph standard can be a deuterium-labeled GlcSph, such as GlcSph(d5) used in the Examples.

Further, in addition to measuring or calibrating the abundance of GlcSph, in some embodiments, the kits are also for use in measuring or calibrating the abundance of one or more bis(monoacylglycero)phosphate (BMP) species (e.g., in a test sample obtained from a subject and/or a reference sample obtained from a reference subject or a population of reference subjects). In some embodiments, the kits comprise a BMP standard (e.g., an internal BMP standard) that can be used for measuring or calibrating the abundance of the one or more BMP species (e.g., in a sample such as a test and/or reference sample). In some embodiments, the BMP standard comprises a BMP species that is not naturally present in the subject. In some embodiments, the BMP standard comprises a BMP species that is not naturally present in humans, non-human primates, rodents, dogs, and/or pigs. In some embodiments, the BMP standard comprises a BMP species that is not naturally present in humans. In some embodiments, the BMP standard comprises BMP(14:0_14:0).

In some embodiments, the kit further comprises one or more reagents. For example, in some embodiments, the kit comprises reagents for obtaining a test sample (e.g., from the subject) and/or a reference sample (e.g., from a reference subject or population of reference subjects), processing a sample (e.g., isolating or purifying GlcSph and/or BMP from a test sample and/or a reference sample), measuring the abundance of GlcSph and/or BMP in a sample (e.g., a test sample and/or a reference sample), and/or calibrating the abundance of GlcSph and/or BMP in a sample (e.g., a test sample and/or a reference sample).

In some embodiments, the kit further comprises instructional materials containing directions (e.g., protocols) for the practice of the methods described herein (e.g., instructions for using the kit of monitoring PGRN levels in a subject (e.g., a test subject and/or a reference subject or population of reference subjects)). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### V. Examples

The following examples are offered for illustrative purposes only, and are not intended to limit the disclosure in any manner.

General Procedure: Quantification of Glucosylsphingosine (GlcSph) in Human Plasma Using LC-MS/MS

To prepare samples for LC-MS/MS analysis, aliquots of 20 μL of calibration standards (STDs), quality control samples (QCs), and unknown plasma samples are transferred to a clean 96-well plate. 20 μL of 1% Bovine Serum Albumin (BSA) in water solution is added to STDs and QCs while 20 μL of methanol is added to plasma samples and plasma matrix QCs. 10 μL of internal standard working solution (5 ng/mL of GlcSph-d5 in acetonitrile/isopropyl alcohol/water 92.5/5/2.5 with 0.5% Formic Acid and 5 mM ammonium formate) is added to each sample followed by 150 μL of 1% ammonium hydroxide in water to adjust pH. After mixing, the samples are loaded onto an ISOLUTE® SLE+ 200 μL Supported Liquid Extraction Plate (Biotage, San Jose, CA). After the samples are completely absorbed in the plate sorbent, ethyl acetate is used to elute the analyte into a clean collection plate. The eluent is dried down completely under purified nitrogen gas flow at 40° C. and reconstituted in 150 μL of acetonitrile/isopropyl alcohol/water 92.5/5/2.5 with 0.5% Formic Acid and 5 mM ammonium formate before the samples are injected to LC-MS/MS for analysis.

LC-MS/MS analyses are performed on an ExionLC AD UHPLC system coupled with a Sciex API 6500 Triple Quad mass spectrometer (AB Sciex, Redwood City, CA). The MRM transitions for GlcSph and GlcSph-d5 are 462.3 to 282.1 and 467.6 to 287.4, respectively. Electrospray ionization was performed in positive ion mode. The Declustering Potential is 60 v and Collisional Energy is 31 v. HPLC chromatography is established on an Acquity BEH HILIC column (1.7 μm, 100×2.1 mm) (Waters Co., Milford, MA) and the column is kept at 55° C. during the run.

For the LC separation of GlcSph from the matrix interference, the two mobile phases used are 0.1% Formic Acid and 10 mM ammonium formate in water (mobile phase A) and 0.1% Formic Acid in acetonitrile (mobile phase B). The flow rate is 0.4 mL/min. Mobile phase B concentration is initially set at 93% and kept for 6.5 min, and then decreased to 50% at 6.51 min and kept for 0.99 min before increased to 93% at 7.51 min. The gradient is ended at 10 min after holding at 93% B for 2.49 min.

### EXAMPLE 1

### GlcSph as a Biomarker for GRN-Linked FTD

Samples from the National Cell Repository for Alzheimer's Disease (NCRAD), which receives government support under a cooperative agreement grant (U24 AG21886) awarded by the National Institute on Aging (NIA), were used in this study. Plasma samples from sporadic FTD patients (n=26), GRN mutant FTD patients (n=16), and clinically normal control subjects (n=20) were obtained.

Plasma samples were blinded, randomized, and subjected to metabolite/lipid extraction by methanol and analyzed on a quantitative LC-MS/MS platform. The analyte identities were confirmed with authentic compounds and each analyte's signal was normalized to a corresponding internal standard. Biomarker data was analyzed by a biostatistician.

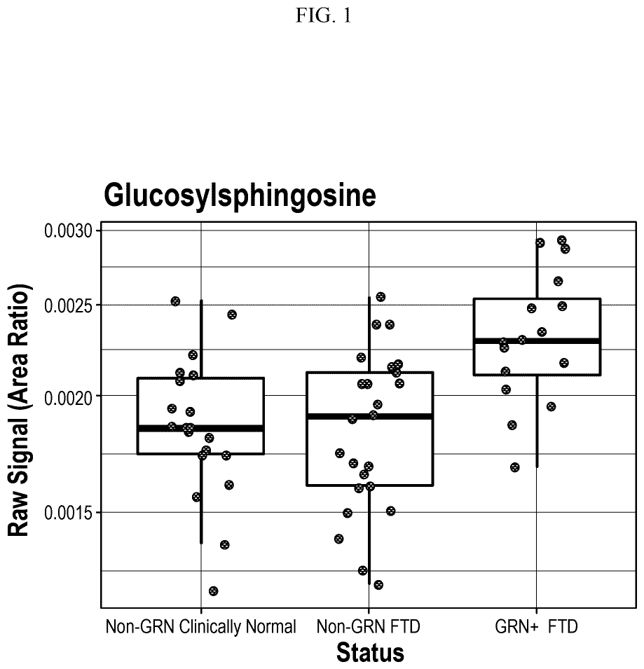

As shown in FIG. 1, there is a significant increase of GlcSph in plasma samples from GRN-linked FTD patients (P<0.01), but not in non-GRNFTD patients, compared to clinically normal controls who do not carry any genetic mutations.

### EXAMPLE 2

### Liquid Chromatography-Mass Spectrometry (LC-MS) Analysis of GlcSph in Various Samples

Tissue samples were weighed (20 mg) and then homogenized in 200 mL methanol spiked with a deuterium-labeled GlcSph, GlcSph(d5), with a TissueLyser homogenizer (Qiagen, Valencia, CA, USA). Homogenates were spun at 14,000 rpm for 20 min at 4° C. Supernatants were then transferred to LC-MS vials for further analysis. The methanol extract was then dried down under nitrogen stream for 4 h and resuspended in 100 μL of 92.5/5/2.5 ACN/IPA/H₂O with 5 mM ammonium formate and 0.5% formic acid for further analyses by LC-MS.

Biofluids (10 μL) were protein-precipitated with 100 μL methanol containing GlcSph(d5) and spun at 14,000 rpm for 20 min at 4° C. Supernatants were then transferred to LC-MS vials for further analysis. The methanol extract was then dried down under nitrogen stream for 4 h and resuspended in 100 μL of 92.5/5/2.5 ACN/IPA/H₂O with 5 mM ammonium formate and 0.5% formic acid for further analyses by LC-MS.

Cells were washed thoroughly with PBS, and GlcSph was extracted with a mixture of water:methanol [1:9, vol:vol] spiked with GlcSph(d5) as internal standard. Samples were vortex mixed and centrifuged at 14,000 rpm for 20 min at 4° C. Supernatants were then transferred to LC-MS vials for further analysis. The methanol extract was then dried down under nitrogen stream for 4 h and resuspended in 100 μL of 92.5/5/2.5 ACN/IPA/H₂O with 5 mM ammonium formate and 0.5% formic acid for further analyses by LC-MS.

GlcSph analyses were performed by liquid chromatography (UHPLC Nexera X2) coupled to electrospray mass spectrometry (QTRAP 6500+). For each analysis, 2-5 μL of sample was injected on a HALO HILIC 2.0 μm, 3.0×150 mm column (Advanced Materials Technology, PN 91813-701) using a flow rate of 0.48 mL/min at 45° C. Mobile phase A consisted of 92.5/5/2.5 ACN/IPA/H₂O with 5 mM ammonium formate and 0.5% formic acid. Mobile phase B consisted of 92.5/5/2.5 H₂O/IPA/ACN with 5 mM ammonium formate and 0.5% formic acid. The gradient was programmed as follows: 0.0-2.0 min at 100% B, 2.1 min at 95% B, 4.5 min at 85% B, hold to 6.0 min at 85% B, drop to 0% B at 6.1 min and hold to 7.0 min, and ramp back to 100% at 7.1 min and hold to 8.5 min. Electrospray ionization was performed in positive or negative ion mode. The following settings were applied: curtain gas at 30 psi; collision gas was set at medium; ion spray voltage at 5500 V; temperature at 400° C.; ion source gas 1 at 50 psi; ion source gas 2 at 60 psi; entrance potential at 10 V; and collision cell exit potential at 12.5 V. Table 2 shows the acquisition parameters and retention time (RT) information for the LC-MS analysis of GlcSph and GalSph species.

GlcSph was identified based on retention time and MRM properties of a commercially available reference standard (Avanti Polar Lipids, Birmingham, AL, USA). Quantification was performed against the internal standard GlcSph(d5) using MultiQuant 3.02 (Sciex). Metabolites were normalized to either total protein amount, tissue weight, or volume.

TABLE 2

| | Internal | | | | Time | | |
|---|---|---|---|---|---|---|---|
| Name | Standard | RT | Q1 m/z | Q3 m/z | (msec) | DP | CE |
| GlcSph | GlcSph(d5) | 6.57 | 462.2 | 282.3 | 200 | 45 | 16 |
| GlcSph(d5) | N/A | 6.58 | 467.2 | 287.3 | 15 | 30 | 16 |
| Galactosylsphingosine | GlcSph(d5) | 6.68 | 462.2 | 282.3 | 200 | 45 | 16 |

### EXAMPLE 3

### Analysis of GlcSph in Plasma of Grn KO/hTfR.KI Mice

Plasma Collection and Processing for Lipid Extraction and GlcSph Analysis

Grn KO/hTfR.KI mice were generated as described in International Patent Publication No. WO 2019/246071.

Fusion 1 was injected via the tail vein into Grn KO/hTfR.KI mice. Following anesthetization of the Grn KO/hTfR.KI mice using a lethal dose of tribromoethanol, whole blood was collected via cardiac puncture using a 1 mL Terumo tuberculin syringe attached to a 25-gauge needle (Ref# SS-01T2516). The blood was then transferred to EDTA-coated tubes (Sarstedt Microvette 500 K3E, Ref# 201341102) and inverted about 10 times. Samples were then spun for 7 minutes at 4° C. and 12700 RPM. Supernatant was then transferred to a new tube with low protein binding and flash frozen. To extract lipids, plasma was thawed on ice, and 10 μL was transferred to 96-well V-bottom half deep-well plates (Waters, Ref# 186005837). 200 μL of the LC-MS grade methanol including internal standards was added to the plasma samples and the plasma samples were shaken at room temperature at 650 rpm for 5 minutes. Samples were then stored at −20° C. for 1 hour to precipitate proteins. After this incubation, plates were spun at 4,000×g for 20 min at 4° C. 50 μL of supernatant was then extracted and transferred to a 96-well plate with glass inserts (Analytical Sales & Services, Ref# 27350). The samples were then dried down under nitrogen steam for about 2 hrs and resuspended in 100 μL acetonitrile/isopropanol/water (92.5/5/2.5, v/v/v) with 5 mM ammonium formate and 0.5% formic acid.

LC-MS Assay for GlcSph

GlcSph analysis was performed by liquid chromatography (Shimadzu Nexera X2 system, Shimadzu Scientific Instrument, Columbia, MD, USA) coupled to electrospray mass spectrometry (Sciex QTRAP 6500+ Sciex, Framingham, MA, USA). For each analysis, 10 μL of sample was injected on a HALO HILIC 2.0 μm, 3.0×150 mm column (Advanced Materials Technology, PN 91813-701) using a flow rate of 0.45 mL/min at 45° C. Mobile phase A consisted of 92.5/5/2.5 ACN/IPA/H₂O with 5 mM ammonium formate and 0.5% formic Acid. Mobile phase B consisted of 92.5/5/2.5 H₂O/IPA/ACN with 5 mM ammonium formate and 0.5% formic Acid. The gradient was programmed as follows: 0.0-3.1 min at 100% B, 3.2 min at 95% B, 5.7 min at 85% B, hold to 7.1 min at 85% B, drop to 0% B at 7.25min and hold to 8.75 min, and ramp back to 100% at 10.65 min and hold to 11 min. Electrospray ionization was performed in the positive-ion mode applying the following settings: curtain gas at 25; collision gas was set at medium; ion spray voltage at 5500; temperature at 350° C.; ion source gas 1 at 55; ion source gas 2 at 60. Data acquisition was performed using Analyst 1.6 (Sciex) in multiple reaction monitoring mode (MRM) with the following parameters: dwell time (msec) and collision energy (CE); entrance potential (EP) at 10; and collision cell exit potential (CXP) at 12.5. GlcSph was quantified using the isotope labeled internal standard GlcSph(d5). Quantification was performed using MultiQuant 3.02 (Sciex).

Results for GlcSph in Plasma

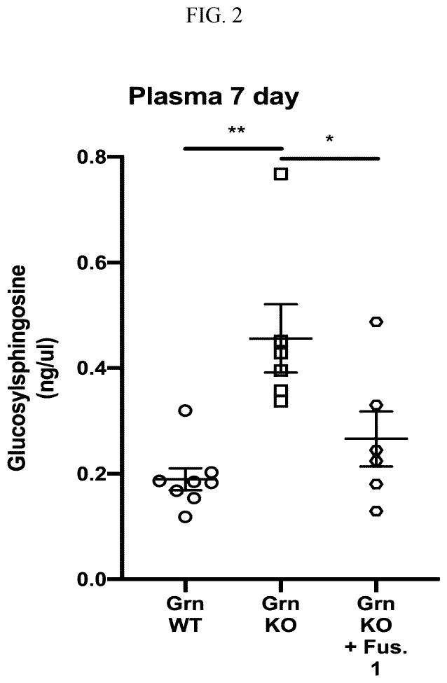

The level of GlcSph was assessed in the plasma of Grn KO/hTfR.KI mice (Grn KO in FIG. 2), littermate controls (Grn WT in FIG. 2), and Grn KO/hTfR.KI mice that received an IV dose of 5 mg/kg Fusion 1 (Grn KO+Fus. 1 in FIG. 2). Fusion 1 is a PGRN derivative containing a PGRN molecule fused to a dimer of Fc polypeptides. Specifically, Fusion 1 contains (1) a PGRN molecule fused to the C-terminus of the first Fc polypeptide in the dimer via a (G₄S)₂ linker, and (2) the second Fc polypeptide in the dimer. The first Fc polypeptide in Fusion 1 is modified with hole mutations T366S, L368A, and Y407V (according to EU numbering scheme) to promote heterodimerization and L234A and L235A mutations (according to EU numbering) to reduce effector function; and the second Fc polypeptide is modified with mutations that enable transferrin receptor (TfR) binding, knob mutation T366W, and L234A and L235A mutations. Fusion 1 is described in detail in International Patent Publication No. WO 2019/246071 (corresponding to Fusion 11 therein), the disclosure of which is hereby incorporated by reference in its entirety.

In Grn KO mice, the GlcSph level in plasma was 2.4 times greater than in Grn WT mice (0.456±0.065 ng/μL & 0.190±0.021 ng/μL, respectively, p=0.002). However, 7 days following Fusion 1 treatment, the GlcSph level in the plasma of Grn KO mice was reduced by 71% compared to the GlcSph level in Grn KO mice not receiving Fusion 1 (0.2661±0.05218 ng/μL, p=0.030).

### EXAMPLE 4

### Analysis of GlcSph in Brain and Liver of Grn KO/hTfR.KI Mice

To determine if the GlcSph upregulation in Grn KO/hTfR.KI mice is specific for plasma or whether it is also found in tissues, in vivo experiments were conducted in Grn KO/hTfR.KI mice dosed with a weekly injection of Fusion 1 or Fusion 2 at 5 mpk for 6 weeks. Fusion 1 is described in Example 3 above and in International Patent Publication No. WO 2019/246071 (corresponding to Fusion 11 therein). Fusion 2 is similar to Fusion 1, except that the second Fc polypeptide in Fusion 2 is not modified to bind to TfR. Fusion 2 contains (1) a PGRN molecule fused to the C-terminus of the first Fc polypeptide via a (G₄S)₂ linker, in which the first Fc polypeptide is modified with hole mutations T366S, L368A, and Y407V (according to EU numbering scheme) to promote heterodimerization and L234A and L235A mutations (according to EU numbering) to reduce effector function (SEQ ID NO:215 in International Patent Publication No. WO 2019/246071), and (2) the second Fc polypeptide modified with knob mutation T366W and L234A and L235A mutations (SEQ ID NO:262 in International Patent Publication No. WO 2019/246071). While Fusion 1 is capable of binding to human TfR, Fusion 2 is not. GlcSph levels in the brain, liver, and plasma of Grn KO/hTfR.KI mice were measured and the ability of Fusion 1 and Fusion 2 to rescue this phenotype was tested.

Plasma collection and preparation for GlcSph analysis were carried out as described in Example 3. Brain and liver tissue collection and preparation for GlcSph analysis are as described below.

Brain and Liver Tissue Collection and Processing for GlcSph Analysis

For tissue LC-MS preparation, during tissue collection, about 20 mg (i.e., 20±2 mg) of brain cortex or liver tissue was dissected, weighed, and flash frozen. 400 μL of methanol spiked with internal standards was added to each sample and homogenized with a 3-mm tungsten carbide bead by shaking at 25 Hz for 30 seconds. The methanol fraction was then isolated via centrifugation at 14,000 g for 20 min at 4° C., followed by transfer of supernatant to a 96-well plate, incubation at −20° C. for 1 h, followed by an additional centrifugation at 4,000 g for 20 min at 4° C., and transferred to glass vials for LC-MS analysis.

For analysis of GlcSph, an aliquot of the methanol fraction was dried under N₂ gas and then resuspended in 100 μL of 92.5/5/2.5 CAN/IPA/H2 (MS grade) with 5 mM ammonium formate (MS grade) and 0.5% formic acid (MS grade). Samples were vortexed, then centrifuged and transferred to glass vials. The LC-MS assay for GlcSph was performed as described in Example 3.

Results for GlcSph in Brain, Liver, and Plasma

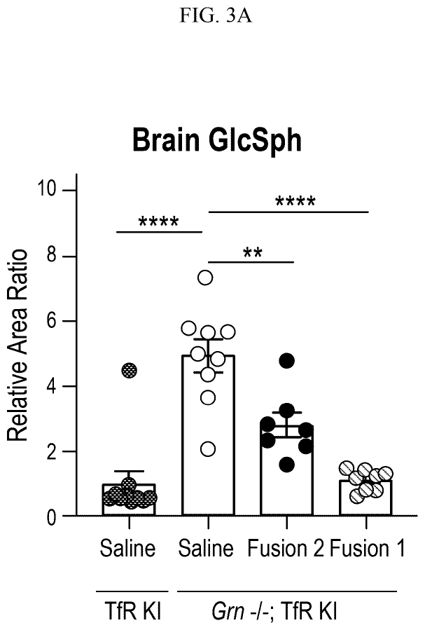

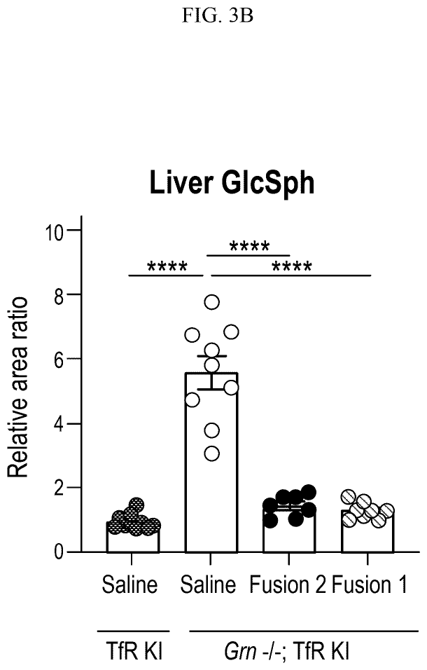

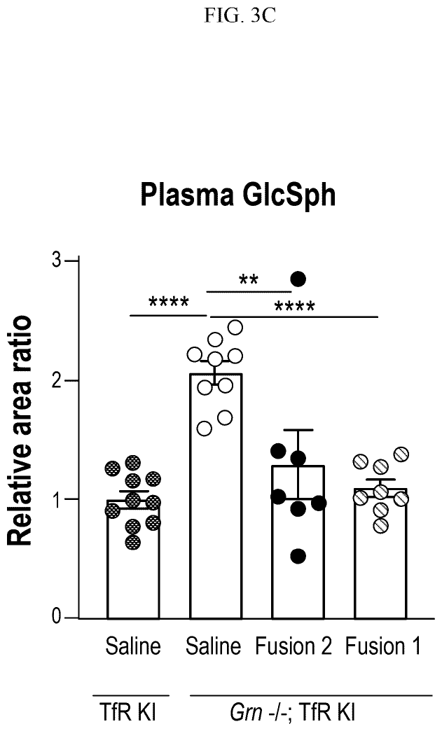

It was found that Grn KO/hTfR.KI mice had higher brain and liver levels of GlcSph compared to control mice, as observed for plasma. After dosing fusion proteins weekly for 6 weeks at 5 mpk, it was found that Fusion 1 fully rescued GlcSph levels in the brain of Grn KO/hTfR.KI mice, whereas Fusion 2 only partially rescued this phenotype (FIG. 3A), indicating that the ability to bind to TfR increases the in vivo efficacy of Fusion 1. In contrast, both Fusion 1 and Fusion 2 were equally able to rescue GlcSph levels in the liver (a peripheral tissue) of Grn KO/hTfR.KI mice (FIG. 3B). Both Fusion 1 and Fusion 2 also rescued GlcSph levels in the plasma of Grn KO/hTfR.KI mice (FIG. 3C).

Together these data show that the increase in GlcSph levels was broadly observed in Grn KO/hTfR.KI mice, including in brain tissue, and that a recombinant PGRN fusion protein capable of binding to human TfR can correct this anomaly in the plasma, liver, and brain of Grn KO/hTfR.KI mice after chronic dosing.

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

1. A method for identifying a subject having, or at risk of having, frontotemporal dementia (FTD), the method comprising:
(a) measuring the abundance of glucosylsphingosine (GlcSph) in a test sample from a subject;
(b) comparing the difference in abundance between the GlcSph measured in (a) and one or more reference values; and
(c) determining from the comparison whether the subject has, or is at risk of having, FTD.

2. The method of claim 1, further comprising administering to the subject a compound for improving the GlcSph level for treating FTD.

3. A method for evaluating a compound or monitoring a subject's response to a compound, pharmaceutical composition, or dosing regimen thereof for treating frontotemporal dementia (FTD), the method comprising:
(a) measuring the abundance of GlcSph in a test sample from a subject having FTD, wherein the test sample or subject has been treated with the compound or pharmaceutical composition thereof;
(b) comparing the difference in abundance between the GlcSph measured in (a) and one or more reference values; and
(c) determining from the comparison whether the compound, pharmaceutical composition, or dosing regimen thereof improves the GlcSph level for treating FTD.

4. The method of claim 3, further comprising treating another test sample or subject with another compound and selecting a candidate compound that improves the GlcSph level.

5. The method of claim 3, further comprising:
(d) maintaining or adjusting the amount or frequency of administration of the compound to the test sample or to the subject; and
(e) administering the compound to the test sample or to the subject.

6. The method of claim 2, wherein the compound is PGRN, a PGRN derivative, or pharmaceutical compositions thereof.

7. The method of claim 2, wherein the compound is a sortilin inhibitor.

8. The method of claim 7, wherein the sortilin inhibitor is an anti-sortilin antibody.

9. The method of claim 1, wherein a subject having, or at risk of having, FTD has an increased GlcSph level compared to the reference value.

10. The method of claim 1, wherein the abundance of the GlcSph in the test sample of a subject having, or at risk of having, FTD is at least about 1.2-fold to about 5-fold higher compared to the reference value.

11. The method of claim 2, wherein the improved GlcSph level is an improvement over the GlcSph level prior to treatment, and wherein the improved GlcSph level is closer to the reference value than the GlcSph level prior to treatment.

12. The method of claim 11, wherein the improved GlcSph level has a difference compared to the reference value of less than 15%, 10%, or 5%.

13. The method of claim 1, wherein the reference value is the GlcSph level in a test sample of the subject prior to the subject receiving treatment.

14. The method of claim 1, wherein the reference value is measured in a reference sample obtained from a reference subject or a population of reference subjects.

15. The method of claim 14, wherein the reference subject or population of reference subjects is a healthy control.

16. The method of claim 14, wherein the reference subject or population of reference subjects does not have FTD or a decreased level of PGRN.

17. The method of claim 1, wherein step (a) further comprises measuring the abundance of one or more bis(monoacylglycero)phosphate (BMP) species.

18. The method of claim 17, wherein the one or more BMP species comprise BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1e_20:4), BMP(20:4_22:6), BMP(18:0e_20:4), BMP(18:2_20:4), BMP(18:1_22:6), BMP(18:1_20:4), BMP(18:0_22:6), and/or BMP(18:3_22:5).

19. The method of claims 1, wherein the test sample or one or more reference values comprise or relate to whole blood, plasma, a cell, a tissue, serum, cerebrospinal fluid, interstitial fluid, sputum, urine, lymph, or a combination thereof.

20. The method of claim 19, wherein the test sample or one or more reference values comprise or relate to plasma.

21. The method of claim 19, wherein the cell is a blood cell, a brain cell, a peripheral blood mononuclear cell (PBMC), a bone marrow-derived macrophage (BMDM), a retinal pigmented epithelial (RPE) cell, an erythrocyte, a leukocyte, a neural cell, a microglial cell, a cerebral cortex cell, a spinal cord cell, a bone marrow cell, a liver cell, a kidney cell, a splenic cell, a skin cell, a fibroblast, a heart cell, a lymph node cell, or a combination thereof.

22. The method of claim 19, wherein the tissue comprises a lymph node, bone marrow, skin tissue, blood vessel tissue, lung tissue, spleen tissue, valvular tissue, or a combination thereof.

23. The method of claim 1, wherein the test sample comprises an endosome, a lysosome, an extracellular vesicle, an exosome, a microvesicle, or a combination thereof.

24. The method of claim 1, wherein the abundance of the GlcSph is measured using liquid chromatography-mass spectrometry (LC-MS), liquid chromatography-tandem mass spectrometry (LC/MS/MS), gas chromatography-mass spectrometry (GC-MS), gas chromatography-tandem mass spectrometry (GC-MS/MS), enzyme-linked immunosorbent assay (ELISA), or a combination thereof.

25. The method of claim 1, wherein an internal GlcSph standard is used when measuring the abundance of the GlcSph.

26. The method of claim 25, wherein the internal GlcSph standard comprises a GlcSph species that is not naturally present in the subject.

27. The method of claim 25, wherein the internal GlcSph standard comprises a deuterium-labeled GlcSph.

28. The method of claims 1, wherein the subject has one or more mutations in the granulin (GRN) gene.

29. The method of claim 1, wherein the FTD is related to PGRN expression, processing, glycosylation, cellular uptake, trafficking, and/or function.

30. The method of claim 1, wherein the FTD is associated with a decreased PGRN level.

31. The method of claim 1, wherein the subject and/or the reference subject is a human or a non-human primate.

32. The method of claim 3, wherein the subject is a PGRN knockout mouse or PGRN knockout rat.

33. A kit for testing a compound or a dosing regimen thereof for treating a FTD in a subject, the kit comprising a GlcSph standard for measuring the abundance of GlcSph in a test sample from the subject.

34-37. (canceled)
